(19) United States
(12) Patent Application Publication
(10) Pub. No.: US 2019/0241849 A1
(43) Pub. Date: Aug. 8, 2019

(54) EXPANDABLE CELL CULTURE SUBSTRATE

(71) Applicant: University of Kansas, Lawrence, KS (US)

(72) Inventor: Adam Joseph Mellott, Parkville, MO (US)

(21) Appl. No.: 16/329,779

(22) PCT Filed: Aug. 30, 2017
(86) PCT No.: PCT/US17/49302

§ 371 Date: Mar. 1, 2019

Related U.S. Application Data
(60) Provisional application No. 62/381,774, filed on Aug. 31, 2016.

(51) Int. Cl. C12M 1/32; C12N 5/00; C12M 3/00; C12M 1/12

(52) U.S. Cl. CPC C12M 23/12; C12N 5/0068; C12M 23/04; C12M 21/08; C12N 5/0062

(57) ABSTRACT

Expandable cell culture substrates are provided that allow continual expansion of adherent stem cells without the need to passage cells, thereby preserving health and stem cell characteristics. The expandable cell culture substrates can include various three-dimensional structures having various coupling mechanisms that allow the structures to be joined, including where the structures are configured as interlocking blocks made from a variety of materials. The three-dimensional structures can include various coupling points, such as protrusions and opposing indentions, to allow for interlocking of the structures in the x-, y-, and z-axial directions. As a cell culture reaches a particular density, additional units can be added to increase the surface area of the expandable cell culture substrate.

### CROSS-REFERENCE TO RELATED APPLICATIONS

This application claims the benefit of U.S. Provisional Application No. 62/381,774, filed on Aug. 31, 2016. The entire disclosure of the above application is incorporated herein by reference.

### FIELD

The present technology relates to an expandable substrate for cell culture, including a substrate that is continuously expandable in one or more dimensions to increase cell culture substrate surface area for the propagation of stem cells.

### INTRODUCTION

This section provides background information related to the present disclosure which is not necessarily prior art.

Adult stem cells have a limited lifespan or period of usefulness following isolation from primary tissues of humans and animals. The act of removing adult stem cells from their original environment can be traumatic to the cells and can have the potential to induce changes in the cells or can have negative effects on the cells. Furthermore, the ability to grow adult stem cells in environments that mimic the original host tissue can be limited, where adult stem cells can lose “stemness” characteristics and the ability to divide and differentiate as the adult stem cells are maintained and dissociated or passaged from various growth substrates, as is current practice. See C. Ng, et al. Biomaterials, 2014; and I. Ullah, et al. Biosci Rep, 2015.

Adherent stem cells can be grown on two-dimensional plastic surfaces (e.g., single or multi-well plates, T-flasks, etc.), in aggregates, or on modified materials for various applications. When adult stem cells cover the majority of the available substrate surface, the cells can start to stratify and form tissue. If tissue formation is not desired, researchers must passage the cells, for example, by chemically and/or enzymatically detaching the cells from the growth substrate, where the cells can be subsequently diluted and plated onto a new growth substrate.

While various passaging techniques provide the ability to expand adult stem cells, such methods can disrupt the physical functions and structural shape of the adult stem cells. As the number of passaging episodes increases, the ability of the adult stem cells to divide and differentiate can diminish, until a point is reached where cells “senesce” (no longer divide) and lose the ability to differentiate. Growth characteristics can also be different in the unnatural two-dimensional environment of a cell culture plate.

Accordingly, there is a need to expand cultured stem cells in a manner that does not lead to differentiation, that does not negatively impact the cells or result in mortality for a portion of the cells, or ultimately induce senescence.

### SUMMARY

The present technology includes systems, processes, articles of manufacture, and compositions that relate to an expandable cell culture substrate, including use thereof in culture of adult stem cells, which can increase the available substrate surface area in one or more dimensions and allow continuous cell propagation without the need for passaging the cells.

Expandable cell culture substrates are provided that include a three-dimensional structure including a coupling point, where the three-dimensional structure also includes a void volume configured to accommodate one or more cultured cells. The coupling point is configured for coupling to another three-dimensional structure. The three-dimensional structure can include a plurality of coupling points. Likewise, there can be a plurality of three-dimensional structures, where each of the three-dimensional structures includes a coupling point and a void volume configured to accommodate one or more cultured cells. The three-dimensional structure can include a porous material having open cells, a reticulated foam-like structure, and/or a mesh-like material. Each of these materials can provide one or more open spaces that sum to provide the void volume of the three-dimensional structure.

Methods of culturing a cell are provided that include providing the cell with one or more expandable cell culture substrates as described herein. Such methods can further include allowing the cell to proliferate in contact with the expandable cell culture substrate. Another expandable cell culture substrate can be coupled to the original expandable cell culture substrate. Cells can then proliferate into contact with the new expandable cell culture substrate coupled to the original expandable cell culture substrate. Available growth surface area can therefore be provided in one-, two-, or three-dimensions.

Methods of making an expandable cell culture substrate are provided that include providing a layer and adding at least one additional layer to the provided layer, thereby forming a plurality of layers. The plurality of layers forms a three-dimensional structure that includes a void volume configured to accommodate one or more cultured cells. The three-dimensional structure is then shaped to form at least one coupling point configured for coupling to another three-dimensional structure.

The expandable cell culture substrate can be configured to be successively expanded in one or more dimensions, where the expandable cell culture substrate enables stem cells to proliferate, expanding the number of the cells many times, where certain situations can provide for indefinitely expanding the cells, all the while minimizing or preventing the loss of key stem cell characteristics. The expandable cell culture substrate can be configured in various shapes and designs, including interlocking three-dimensional structures, as described herein, which are simple to use, provide multiple options for different applications (e.g., different materials, sizes, shapes, additives, etc.), and can reduce the cost of consumables and time required for cell culture maintenance, including the propagation of stem cells. In this way, the present technology can prevent or reduce the number of stem cells that undesirably differentiate in culture, can eliminate negative effects on the cells resulting from passaging the cells, and can further prevent senescence and loss of pluripotency.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a perspective view of an expandable cell culture substrate, where the substrate includes a three-dimensional structure having two coupling points.

FIG. 2 is a cross-sectional view of the expandable cell culture substrate of FIG. 1 taken along plane 2-2.

FIG. 3 is perspective view of two expandable cell culture substrates of FIG. 1 coupled together.

FIG. 4 is a cross-sectional view of the coupled expandable cell culture substrates of FIG. 3 taken along plane 4-4.

FIG. 5 is a perspective view of another expandable cell culture substrate.

FIG. 6 depicts perspective views of various expandable cell culture substrates that are coupled in various configurations, where panel A shows various separate substrates that can be assembled to form the various coupled substrate groups in panels B and C.

FIG. 7 depicts a 6-well cell culture plate, each well including a different expandable cell culture substrate, where each well/substrate is seeded with cells.

FIG. 8 depicts a cell culture plate including an expandable cell culture substrate that is at or near saturation with cells coupled to a newer expandable cell culture substrate, allowing the cells to proliferate and expand into the newer expandable cell culture substrate.

FIG. 9 depicts a series of photomicrographs of an expandable cell culture substrate having cells proliferating in the void volume thereof, where panel A shows about 3.5 million cells at day 15, panel B shows about 88 million cells at day 30, and panel C shows about 361 million cells at day 60.

FIG. 10 is a schematic of a cell culture apparatus using expandable cell culture substrates coupled in three dimensions to grow stem cells in a manner that does not lead to differentiation, that does not negatively impact the cells or result in mortality for a portion of the cells, or ultimately induce senescence.

FIG. 11 depicts a method of making an expandable cell culture substrate using multiple layers of material.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments. Except where otherwise expressly indicated, all numerical quantities in this description are to be understood as modified by the word “about” and all geometric and spatial descriptors are to be understood as modified by the word “substantially” in describing the broadest scope of the technology. “About” when applied to values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and “substantially” as used herein indicates at least variations that may arise from ordinary methods of measuring or using such parameters.

All documents, including patents, patent applications, and scientific literature cited in this detailed description are incorporated herein by reference, unless otherwise expressly indicated. Where any conflict or ambiguity may exist between a document incorporated by reference and this detailed description, the present detailed description controls.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, all compositional percentages are by weight of the total composition, unless otherwise specified. Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

The present technology overcomes issues that limit stem cell production and expansion by providing an expandable and transferable cell culture substrate that can enable indefinite stem cell growth. Current technologies rely on expanding cells through dissociation. The present expandable cell culture substrates can save time and resources by reducing the materials and steps required for cell culture maintenance and expansion while enabling the expansion of high quality stem cells at low passage numbers. Reference to cultured cells herein are not limited by cell type and are expressly intended to include stem cells, as well as adult stem cells.

In certain embodiments, an expandable cell culture substrate is provided that includes a three-dimensional structure having a coupling point. The three-dimensional structure includes one or more interior spaces forming a void volume that is configured to accommodate one or more cultured cells. The coupling point is configured for coupling to another three-dimensional structure. For example, the three-dimensional structure and the another three dimensional structure can be the same, where the structures can be coupled through identical coupling points, different coupling points, or complementary coupling points. The three-dimensional structure and the another three-dimensional structure can also be different, but can be coupled through identical coupling points, different coupling points, or complementary coupling points. Expandable cell culture substrates allow for cell culture in three dimensions, where the substrate can be expanded in x-, y-, and z-axial directions, for example, by coupling one three-dimensional structure to another three-dimensional structure.

With respect to the coupling point, the three-dimensional structure can have a single coupling point or the three-dimensional structure can have a plurality of coupling points. Each coupling point in the plurality of coupling points can be the same, different, or a combination of identical and different coupling points. For example, the plurality of coupling points can include a first coupling point and a second coupling point. The first coupling point can be configured to couple to the second coupling point. The first coupling point can be a male coupling point and the second coupling point can be a female coupling point, where the male coupling point and the female coupling point are complementary. That is, the male coupling point is configured to be received by the female coupling point. Other examples of such coupling points include various shaped protrusions and various shaped recesses.

Coupling points can be configured with various coupling architectures allowing the coupling of additional three-dimensional structures to expand the available growth surface area, allow the replacement of one or more three-dimensional structures, or permit the removal of one or more three-dimensional structures from a culture. Further examples of coupling between three-dimensional structures of the expandable cell culture substrate include various interlocking and reversible coupling points, male-female fittings including mortise and tenon, tongue-and-groove, dovetails, cross laps, magnetic couplings, and various reactive or chemical couplings. Coupling points can be reversible, allowing separation of coupled three-dimensional structures, or coupling points can be irreversible, where the coupling point or another portion of the three-dimensional structure would be damaged in attempting to separate the coupled three-dimensional structures. For example, a three-dimensional structure can have a coupling point having a hook or barb that pierces or penetrates another three-dimensional structure. Three-dimensional structures with coupling points including various protrusions and recesses can be configured as interlocking blocks. Assembly and coupling of multiple three-dimensional structures together can take the form of various tessellations and can resemble the assembly of jigsaw pieces in one, two, or three-dimensions.

The expandable cell culture substrate can include a plurality of three-dimensional structures. Each three-dimensional structure in the plurality of three-dimensional structures can include a coupling point and a void volume configured to accommodate a cultured cell. The coupling point of each three-dimensional structure can be configured for coupling to another three-dimensional structure. The plurality of three-dimensional structures can comprise all of the same type of three-dimensional structure or there can be multiple types of three-dimensional structures. The plurality of three-dimensional structures can include separate three-dimensional structures and/or two or more three-dimensional structures already coupled together.

In certain embodiments, the expandable cell culture substrate is configured as three-dimensional structures of various shapes and sizes that can be coupled together or uncoupled from each other. The expandable cell culture substrate can include repeating units that are configured to be coupled together in various ways, permitting expansion of the substrate in various dimensions, including expansion in three dimensions. Single types of three-dimensional structures or multiple types of three-dimensional structures can be used. For example, identical three-dimensional structures can have coupling features that allow expansion in one or more dimensions, where the dimensions can be defined by x-, y-, and z-axial directions. Taking a single axial direction, for example, the expandable cell culture substrate can include a three-dimensional structure having a male coupling feature on one end of an x-axis of the unit and a female coupling feature on the other end of the x-axis of the unit. In this way, multiple three-dimensional structures (represented by “A”) can be linearly coupled along the x-axis to form a compound structure, where two such three-dimensional structures can form the compound structure “A-A,” three such units could form the compound structure “A-A-A,” and so on. Successive or later addition of three-dimensional structure(s) “A” can provide a structure of “A_{N},” where N=the number of units. Similar expansion of the cell culture substrate can occur in other dimensions where the three-dimensional structures employed include coupling features in other dimensions; e.g., successive addition of units along the y-axis and/or z-axis. It is also contemplated that multiple types of three-dimensional structures can be employed, where a unit represented by “A” can have a coupling feature that is not compatible with itself, but is compatible with a coupling feature of a different unit represented by “B.” Again, taking a single axial direction as an example, these units can be coupled to form the structures “A-B,” “A-B-A,” “A-B-A-B,” and so on. Coupling of various three-dimensional structures of the expandable cell culture substrate can therefore expand multiple three-dimensional structures or particular three-dimensional structures in one or more various dimensions, including expanding the cell culture substrate in three dimensions.

Three-dimensional structures of the expandable cell culture substrate can have various shapes, sizes, and dimensions. In this way, the expandable cell culture substrate can provide additional cell culture surface area as needed, can be configured to fit within various cell culture vessels and bioreactors, and can be expanded in one or more directions to fit within vessels and bioreactors of predetermined shapes and sizes. The three-dimensional structure can be formed with various shaped facets or curved planes, where examples include various polyhedral, cylindrical, conical, spherical, ovoid, bead-like, and fiber-like structures. Certain embodiments include three-dimensional structures formed as hexahedrons including cubes and cuboids. One or more coupling points can be formed in the three-dimensional structure as part of the overall structure or in addition to the overall structure. Examples of three-dimensional structures include those having sub-millimeter scale dimensions, millimeter scale dimensions, up to dimensions approaching the centimeter scale. For example, three-dimensional structures can have dimensions ranging from 0.1 to 10 millimeters in each of the x-, y-, and z-axial directions. Other three-dimensional structures can have dimensions ranging from 0.5 to 10 millimeters in each of the x-, y-, and z-axial directions. Certain embodiments of three-dimensional structures include cuboids that are about 10 mm by 10 mm by 10 mm in x-, y-, and z-dimensions with about 2.5 mm by 2.5 mm by 2.5 mm cube-shaped protrusions and opposing about 2.5 mm by 2.5 mm by 2.5 mm cube-shaped indentions as coupling points that can allow coupling or interlocking of the expandable cell culture substrates in the x-, y-, and z-axial directions.

In some embodiments, the expandable cell culture substrate is configured as various individual units or pre-coupled units defining various three-dimensional structures. It should be understood that various other configurations of expandable cell culture substrates as provided by the present technology can be configured and can be used in similar ways as described herein. That is, the details pertaining to the examples of three-dimensional structures provided herein can be adapted to various other shapes, sizes, unit numbers, materials, and mixtures of various homogeneous and heterogeneous expandable cell culture substrates.

The expandable cell culture substrate can include a three-dimensional structure formed of a variety of different synthetic and biological materials. One or more materials used in the expandable cell culture substrate can be modified to change chemical attributes (e.g., pH, electrical charge, surface moieties, etc.) or mechanical attributes (e.g., biodegradable, stable, thermal stability, physical strength, elasticity, etc.) to suit different conditions for isolating and culturing different cell types. Expandable cell culture substrates can be infused with additional materials to promote or inhibit cell growth to model injuries or diseases at a cellular level. Examples of materials that can be used to form the three-dimensional structures of expandable cell culture substrates include poly(lactic-co-glycolic acid), polylactic acid, polycaprolactone, hyaluronic acid, chitosan, alginate, acellular matrix, polyurethane, polyurethane-polyether, polycarbonate, and combinations thereof. Examples of additives that can be included in the three-dimensional structure are metals such as silver and gold, fibronectin, chitosan, poly-L-lysine, hyaluronic acid, one or more growth factors, laminin, fibrin, and combinations thereof.

In certain embodiments, the three-dimensional structure can include poly(lactic-co-glycolic acid) (PLGA), where PLGA is an FDA approved biomaterial that is biodegradable and can be modified for a variety of applications. PLGA can be formatted into different shapes such as fibers. Expandable cell culture substrates can be manufactured from PLGA non-woven and woven mesh fiber sheets, including those available from Biomedical Structures, Warwick, R.I. Polylactic acid (PLLA) is another biocompatible and biodegradable polymer that can be formatted into different shapes. Expandable cell culture substrates can be manufactured from PLLA non-woven and woven mesh fibers, also available from Biomedical Structures, Warwick, R.I. Polycaprolactone (PCL) is yet another biocompatible and biodegradable polymer that has been approved for specific drug-delivery applications within the human body. PCL can be formatted into different shapes such as fibers and spheres via electrospinning or precision particle fabrication, respectively. PCL is available from Sigma-Aldrich, St. Louis, Mo. Hyaluronic acid, chitosan, and alginate hydrogels can also be used to fabricate expandable cell culture substrates. Decellularized and devitalized acellular matrices can be cut and formed into expandable cell culture substrates. Encapsulated cells in a hydrogel formulation can be formed into an expandable cell culture substrate. Other materials useful in forming expandable cell culture substrates include biomaterials of woven and non-woven fabrics formed of polymer fibers made of PLGA, PLLA, and/or PCL with varying thicknesses and molecular weights.

Additional examples of materials that can be used alone or in combination to form expandable cell culture substrates include various synthetic materials, natural materials, combinations thereof, and modified versions thereof. Synthetic material examples include polyurethane (PU), polyurethane polyether, polyurethane polyester, polyamide (PA), polyvinyl chloride (PVC), polyethylene (PE), polypropylene (PP), polystyrene (PS), polytetrafluoroethylene (PTFE), and polychlorotrifluoroethylene (PCTFE). Various types of natural and modified collagens can be used, including: collagen (All types and Sub-types), collagen type I (COL1A1, COL1A2), collagen type II (COL2A1), collagen III (COL3A1), collagen type IV (COL4A1, COL4A2, COL4A3, COL4A4, COL4A5, COL4A6), collagen type V (COLA1, COL5A2, COL5A3), collagen type VI (COL6A1, COL6A2, COL6A3, COL6A5), collagen type VII (COL7A1), collagen type VIII (COL8A1, COL8A2), collagen type IX (COLA9A1, COLA9A2, COL9A3), collagen type X (COL10A1), collagen type XI (COL11A1, COL11A2), collagen type XII (COL12A1), collagen type XIII (COL13A1), collagen type XIV (COL14A1), collagen type XV (COL15A1), collagen type XVI (COL16A1), collagen type XVII (COL17A1), collagen type XVIII (COL18A1), collagen type XIX (COL19A1), collagen type XX (COL20A1), collagen type XXI (COL21A1), collagen type XXII (COL22A1), collagen type XXIII (COL23A1), collagen type XXIV (COL24A1), collagen type XXV (COL25A1), collagen type XXVI (COL26A1), collagen type XXVII (COL27A1), collagen type XXVIII (COL28A1). Other materials include chitosan, alginate, acellular matrix, and nucleic acids.

The three-dimensional structure of the expandable cell culture substrate can provide a void volume to accommodate a cultured cell in various ways. For example, the three-dimensional structure can be formed of woven and/or non-woven fibers that provide a lattice or scaffold-like structure, where the open space between the fibers provides the void volume. Size and density of such fibers can be tailored to provide a desired void volume having interstitial dimensions to accommodate cells of certain sizes and shapes. The void volume can also be formed from a porous material having open cavities, where cultured cells can proliferate and expand between the open or interconnected pores. The three-dimensional structure can also formed as a reticulated foam or a mesh-like material, which can accommodate tensile, compression, and shear forces.

Expandable cell culture substrates can be formed with or can be impregnated with one or more various materials, where the three-dimensional structure can be formed, soaked, or filled with a solution including a suspended or dissolved material. The expandable cell culture substrate can be infused, impregnated, or coated with these additional materials in order to promote or inhibit cell growth to model injuries or diseases at a cellular level. Various additives for expandable cell culture substrates include silver nanoparticle impregnation (e.g., antimicrobial properties), gold coating (e.g., electric conduction for electroporation), fibronectin coating, chitosan coating, poly-L-lysine coating, hyaluronic acid infusion, and one or more growth factors.

In particular, expandable cell culture substrates can be soaked with an aqueous solution containing silver nanoparticles to impregnate the three-dimensional structure with silver nanoparticles to impart antimicrobial properties. The silver nanoparticles can be impregnated into a variety of manufactured materials to impart antimicrobial or electroconductive properties to materials used to form the expandable cell culture substrate. Gold can be sputter-coated on materials to make a material conductive for electron microscopy applications and electroporative applications for gene delivery. Similar to impregnation, expandable cell culture substrates that are porous (e.g., woven and non-woven meshes) can be soaked in a laminin or fibrin solution to allow for fibers to be coated with laminin or fibronectin to enable the attachment of primary stem cells through integrin binding of extracellular components to laminin or fibronectin. Laminin is found in the basement membrane of several connective tissues and facilitates cell binding and migration. Fibronectin is found in the serum of the blood and contains several moieties that contain polypeptides that facilitate attachment of mammalian cells via integrin binding. Fibronectin can be coated on materials to therefore promote cell attachment and migration. Poly-L-lysine is an amino acid polymer that can be used to coat materials to promote cell attachment. Poly-L-lysine can be used to promote binding of neural cells. Hyaluronic acid is a major component found in the extracellular matrix of cartilage, bone, and neural tissues. Hyaluronic acid can be included to promote cell attachment and cell differentiation. Cell growth factors, including various proteins, agents, and cytokines that can activate cell pathways that lead to protein production and cell differentiation, can also be included. These can include TGFβ, BMP neurotrohphins, SOX9, SOX2, OCT4, and NANOG, among others.

The expandable cell culture substrates can further include various chemicals, pharmaceuticals, proteins, and/or viral particles. In this way, the cells can be grown in culture in the presence of one or more of these additives, where proliferating cells are provided with increased surface area and are exposed to the one or more additives. Adding one or more new expandable cell culture substrates to the culture including a different cell culture substrate material and/or having different additives can also provide a challenge or selective condition upon the proliferating cells. For example, cells from a primary tissue sample can grow into one or more expandable cell culture substrates, where subsequent addition of new expandable cell culture substrates including a chemical agent can limit expansion onto the new expandable cell culture substrates to cells that are capable of tolerating the chemical agent or are attracted to the chemical agent. Different selective conditions or pressures can therefore be applied to the cell culture at different stages by the subsequent addition of one or more expandable cell culture substrates.

Expandable cell culture substrates can be configured to be compatible with a variety of standard tissue culture vessels and such substrates can be used in a variety of bioreactors. Examples of cell culture with expandable cell culture substrates include using the substrates in various cell culture plates, where one or more additional expandable cell culture substrates can be added thereafter to provide additional surface area for cultured cells, as necessary. Various cell culture plates include 6-well plates, 12-well plates, 24-well plates, 48-well plates, 96-well plates, 30 mm mono-plates, 60 mm mono-plates, 100 mm mono-plates, 150 mm mono-plates, etc. Expandable cell culture substrates can therefore allow the continual expansion of adherent stem cells without the need to passage the cells, thereby preserving health and stem cell characteristics. The expandable cell culture substrate can also be used to freeze high-quality primary cells in low passage numbers at high cell densities.

Various methods can be used to form expandable cell culture substrates, where various substrates can be molded, cast, or assembled by various iterative processes. Forming processes can include extrusion, drawing, wet spinning, melt spinning, and electrospinning. Extrusion is a method by which a material is forced or pushed through an opening or nozzle to create an object with a fixed cross-sectional profile, such as a fiber. Drawing is where a material is pulled through an opening to create an object with a fixed cross-sectional profile, such as a fiber. Wet spinning can be used to form fibers, where polymers can be dissolved in a solvent and spun using a spinneret. The spinneret can be submerged in a chemical bath that causes the polymer to form a fiber as it precipitates through the spinneret and solidifies. Other common manufacturing techniques to produce fibers can be used. Melt spinning is similar to wet spinning, except polymers are melted and extruded from a spinneret, where polymers solidify as they dry and cool. Electrospinning is similar to wet spinning and melt spinning, where electric force is used to draw polymers that are dissolved in solvent or melted through a spinneret to form very fine fibers that have diameters in the micrometer range. Polymer fibers can be woven using a custom loom or electrospinning to weave liquid polymers in custom patterns that closely resemble target tissue extracellular matrix architecture. Briefly, polymers can be melted down or dissolved and extruded out of a nozzle to form fibers that are fed into a custom loom to weave a custom textile. Such expandable cell culture substrates can be formed from a variety of different materials (e.g., polymers, hydrogels, acellular matrices, etc.) depending on the need or application. These substrates can be fabricated from various materials using various methods, including the overlay of various woven and/or non-woven fibers.

In certain embodiments, methods of making an expandable cell culture substrate are provided. A layer of material is provided where at least one additional layer of material is added to the provided layer of material. A plurality of layers of material are thereby formed, where the plurality of layers of material provides a three-dimensional structure including a void volume configured to accommodate a cultured cell. Several additional layers of material can be added, where each layer has dimensions in the x- and y-axes and the successive addition of layers increases a dimension of the three-dimensional structure in the z-axis, for example. The three-dimensional structure can be shaped to form one or more coupling points configured for coupling to another three-dimensional structure. The shaping can also modify the overall size and shape of the three-dimensional structure and can adapt individual three-dimensional structures as interlocking blocks, including sizes and shapes to form various tessellations with the three-dimensional structures. The various layers can be formed entirely of or include the same material or different materials. For example, alternating layers or various repeating or non-repeating patterns of different fibers can be used to form the three-dimensional structure. Fiber size, fiber spacing, fiber material, and additives can be tailored or optimized for each layer depending on the desired characteristics for the resulting three-dimensional structure. Additional layers can also be added at various orientations, including an orientation that is different from the previous layer. The character of the fiber network, lattice, or scaffolding can therefore be controlled in this fashion, where the void volume of the three-dimensional structure can include continuous and substantially straight or direct vias that are maintained through the three-dimensional structure. In addition to or conversely, the void volume can be configured to have tortuous pathways throughout the three-dimensional structure by overlaying fibers of one or more successive layers to cover spaces in one or more preceding layers.

Various fibers within each layer and/or various fibers between layers can be coupled together in various ways. The fibers can be configured to interlock, having hook and loop characteristics for example, or the fibers can be chemically cross-linked using a cross-linking agent or heat. In certain cases, the layers of the three-dimensional structure can be sintered using heat or joined by partially dissolving fibers or layers with solvent which is then removed.

Shaping of three-dimensional structure can be performed in various ways. For example, a three-dimensional structure can be built up using successive layers in the z-axis, where the overall dimensions of the layers in the x- and y-axes are substantially larger than the z-axis dimension, giving the three-dimensional structure an overall plate-like configuration. The three-dimensional structure can be cut or shaped to produce multiple smaller three-dimensional structures from the large plate-like precursor. In certain embodiments, the three-dimensional structure can be die cut to provide a plurality of three-dimensional structures. The die can include various interlocking patterns, including those resembling jigsaw puzzle patterns, thereby defining how the individual three-dimensional structures can be coupled together in a predefined arrangement or tessellation.

Some embodiments of three-dimensional structures include layering or molding non-woven sheets of poly(lactic-co-glycolic acid) (PLGA), poly(lactic acid) (PLA), and/or poly(L-lactide) (PLLA) into various three-dimensional structures. PLGA, PLA, and/or PLLA can be suitable to support growth of primary cells. The fiber weave can also be engineered to mimic and substantially match the native tissue architecture from which the cells originate.

Other additive manufacturing processes for expandable cell culture substrates include the use of 3D printing, which can include melting and extruding a polymer out of a predefined nozzle, such as a 100 μm diameter nozzle, and layering the polymer in a pre-defined shape. As the polymer is extruded and layered, the three-dimensional structure of the expandable cell culture substrate can be formed and manufactured according to custom parameters. An advantage of using 3D printing is that thermopolymers such as PLGA or PLLA can be used in addition to custom hydrogels (e.g., alginate, hydroxyapatite, etc.) where cells can be pre-encapsulated in the three-dimensional structure of the expandable cell culture substrate, depending on a particular application.

Woven and non-woven materials can also be blended with different polymer types. Expandable cell culture substrates can be cut by hand, or by using a mechanical press, into various shapes, including 1 cm cubes with 0.25 cm complimentary protrusions and sockets, for example. Commercially manufactured textiles such as woven and non-woven meshes can be used for cutting expandable cell culture substrates. A mesh or screen can be used as a die to simultaneously cut multiple expandable cell culture substrate faces. For example, a sheet of material can be cut by a die or press to form a single expandable cell culture substrate, or the die can use a mesh or screen to cut multiple expandable cell culture substrates from the sheet of material.

Various post-forming processes can be applied to expandable cell culture substrates, including sterilizing methods, curing steps, and cross-linking steps. Expandable cell culture substrates can be sterilized via UV irradiation. Treatment of expandable cell culture substrates with a 24 hour exposure to ethylene oxide can also provide sterilization of the expandable cell culture substrate. Various curing or cross-linking agents can be used, including where the expandable cell culture substrates include a photosensitive material, for example.

Expandable cell culture substrates can be used in various ways. Methods of culturing one or more cells, including stem cells, are provided. One or more cells are provided with an original expandable cell culture substrate. The one or more cells can be allowed to proliferate. One or more expandable cell culture substrates can then coupled to the original expandable cell culture substrate. In this manner, proliferating cells can grow into and contact the additional expandable cell culture substrate(s). Additions of expandable cell culture substrates can occur in various dimensions. The expandable cell culture substrate can also be perfused with cell culture media, allowing continuous or timed replenishment of growth media. The expandable cell culture substrate can also be transferred to another culture vessel, plate, or bioreactor, where there is no need to dissociate the cells growing in contact with the expandable cell culture substrate. Cells from various cultures and/or sources can therefore be combined by coupling of expandable cell culture substrates. One or more expandable cell culture substrates can be obtained that have a desired confluence or populated with a desired number of cells. Likewise, asymmetrical cell populations within expandable cell culture substrates can be prepared, including expandable cell culture substrates having cell density gradients therethrough.

Expandable cell culture substrates can be placed into contact with a tissue explant that has been removed from a host organism, such as a human or animal. The tissue explant and expandable cell culture substrate can be cultured together. The expandable cell culture substrate therefore provides new surface area for cells to migrate onto from the tissue explant. Cells migrating from the tissue explant include propagating cells. After cells have migrated into the expandable cell culture substrate, the tissue explant can be discarded or used for other applications. As cells divide and cover the surface area within the expandable cell culture substrate, one or more additional expandable cell culture substrates can be added and coupled to the original expandable cell culture substrate in the x-, y-, or z-axial directions to provide additional surface area. As cells continue to divide and occupy available surface area, additional expandable cell culture substrates can be coupled as needed to provide additional surface area. The number of expandable cell culture substrates can be increased to fill the cell culture vessel or bioreactor. The entire collection of expandable cell culture substrates can be transferred to a larger cell culture vessel or bioreactor, as needed, or one or more units or portions of the coupled expandable cell culture substrates can be decoupled or removed and transferred to one or more new cell culture vessels or bioreactors. Use of the expandable cell culture substrate allows investigators to increase cell culture surface area as necessary without passaging cells, thus saving time and preserving original cell characteristics.

Various types of cells and tissue explants can be cultured in contact with the expandable cell culture substrate. Cells can be selected from human cells or other mammalian species. Examples include various types of stem cells, including pluripotent stem cells and adult stem cells. Pluripotent stem cells include embryonic and induced pluripotent stem cells. Adult stem cells include mesenchymal stem cells (derived from bone marrow, adipose tissue, dermis, and umbilical cord), fibroblasts, neuro-stem cells, endothelial cells, and progenitor cells (neuro, vascular, dermal, muscular, skeletal, cartilage including chondroblasts, bone including osteoblasts, intestinal, and cardiac). Other cells include cancer cells isolated in various ways, including biopsies, blood draw, tissue explants, established cell culture lines derived from cancerous cells, etc. Various somatic cells can be used, including neurons, glial cells, Schwann cells, oligodendrocytes, astrocytes, hepatocytes (liver cells), chondrocytes (cartilage), adipocytes (fat), osteocytes (bone), osteoclasts (bone), keratinocytes, skeletal muscle cells, smooth muscle cells, cardiac muscle cells. Various blood cells can be used, including red blood cells and white blood cells, where white blood cells include macrophages, neutrophils, eosinophils, basophils, lymphocytes, monocytes, B cells, T Cells, natural killer cells (NK cells). Still further types of cells can be selected from neurosensory cells, including the inner ear, eyes, and the nasal lining.

Expandable cell culture substrates can be perfused with solutions of different viscosities and compositions to mimic native environments or to induce flow of nutrients through the three-dimensional structure of the expandable cell culture substrate. If a substantial number of expandable cell culture substrates are joined together, then a perfusion bioreactor can be used to facilitate movement of culture media therethrough to supply nutrients to all cells located within substrates that no longer have exposed surfaces.

Expandable cell culture substrates can further include the following aspects. Expandable cell culture substrates can be used to isolate primary adult stem cells from host tissues. Expandable cell culture substrates can be used to indefinitely expand primary cells. Expandable cell culture substrates can be used to preserve original stem cell characteristics (e.g., CD markers, differentiation potential, division, etc.) or maintain low passage, high quality stem cells. Expandable cell culture substrates can be used to isolate and culture primary cells (e.g., neurons, cancer cells, muscle cells, etc.) in addition to stem cells to grow and model healthy and diseased tissues outside of the body for study. Expandable cell culture substrates can be used to grow cells, tissues, or organoids for drug testing, drug delivery, and/or for implantation. Expandable cell culture substrates can also be used as models for development of new bioengineering therapies.

The expandable cell culture substrates can be used in conjunction with many established tissue culture techniques and methods. As will be recognized by one skilled in the art, methods relating to cell isolation, maintenance, dissociation, freezing, and transfection can take advantage of the present technology. To illustrate, the following general cell culture protocols can be employed.

With respect to cell isolation, native tissue biopsies can be cut into 0.5 cm² squares and washed twice with sterile 2% antimycotic and antifungal solution in phosphate buffered saline (PBS). Biopsies can then be soaked in migration media (5 mL sterile 1% collagenase, 10% Fetal Bovine Serum (FBS)) and placed on top of the flat sides of fibronectin coated expandable cell culture substrates and incubated at 37° C. for 10 days, with migration media changes every two days. After 10 days, the expandable cell culture substrates can be washed with sterile PBS three times, and soaked in maintenance media (10% FBS in DMEM).

With respect to cell maintenance, maintenance media can be changed every two days, and when cells had covered 60% of the observable expandable cell culture substrate surface area, a second expandable cell culture substrate coated in fibronectin can be coupled to the original. Expandable cell culture substrates can then be expanded as needed within open-well plates.

With respect to cell dissociation, cells can be dissociated from the expandable cell culture substrates either by using traditional chemical methods, such as breaking integrin bonds using trypsin, or by degrading or dissolving the three-dimensional structure of the expandable cell culture substrates with enzymes or solvents that degrade the material forming at least a portion of the three-dimensional structure.

With respect to cell storage or freezing, expandable cell culture substrates infused with cells can be flash-frozen in freezing media (10% dimethyl sulfoxide in DMEM) and stored in liquid nitrogen. Frozen expandable cell culture substrates can be thawed in 37° C. warmed PBS, and transferred into thawing media (10 μM Y-27632 Rock inhibitor, 20% FBS, DMEM) for 24 hours. After 24 hours, the expandable cell culture substrates can be transferred into maintenance media.

With respect to cell transfection, expandable cell culture substrates can be sputter-coated with gold to provide electroconductive properties, then sterilized via ethylene oxide, and coated with fibronectin. Cells can then be seeded on the expandable cell culture substrates, and cultured until cells covered 60% of observable fiber surface area. The expandable cell culture substrate can then be connected to a positive and negative electrode. The cell-infused expandable cell culture substrate can be soaked in a saline solution with 10 μg of Green Fluorescent Protein (GFP) plasmid DNA. A 5 ns pulse of 1 mV can be applied to the cell infused substrate, and cells can be imaged for GFP signal after 24 hours.

The present technology can also be employed in various ways, including drug testing, organiod engineering, tissue engineering, protein production, stem cell expansion, primary cell expansion, genetic manipulation and transformation. Cell culture and bioengineering have focused on the culture of cells and implanting the cultured cells back into tissues. The present technology provides a special technical effect, as the present technology allows “reverse cell culture” where the expandable cell culture substrate is designed to isolate cells and continue growing cells as an artificial removable extension of the host, thus preserving the state of the cells or disease as the sample is transferred to a culture environment for various analyses, diagnostics, treatments, and/or modifications. The present technology solves the surface area problem regarding adult stem cells and allows propagation of such cells without loss of pluripotency.

### EXAMPLES

With reference to the several figures, the following examples demonstrate aspects of the present technology.

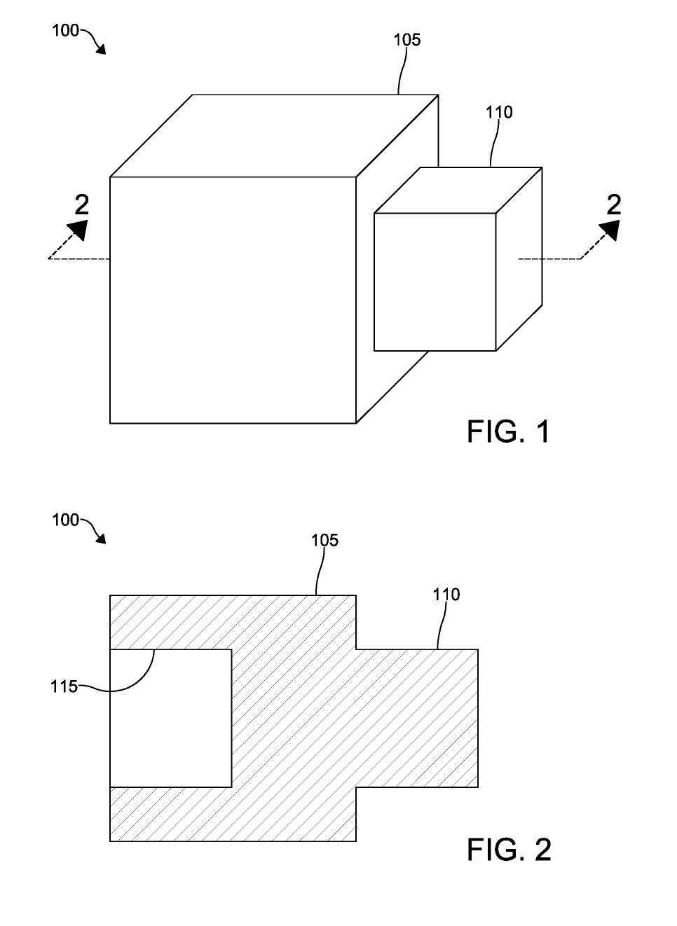

FIGS. 1 and 2 show a first expandable cell culture substrate 100 including a three-dimensional structure 105 having a male coupling point 110 and a female coupling point 115. It can be seen that the male coupling point 110 is formed as a cuboid protrusion from the three-dimensional structure 105. The female coupling point 115 is formed as a cuboid recess into the three-dimensional structure 105. The three dimensional structure 105 includes a void volume (not shown) comprising the sum of open space within the three dimensional structure 105. The three dimensional structure 105 is depicted as a generally cuboid structure, but should be considered to have interconnected pores therein, such as that found in a sponge or a reticulated foam structure, or where the three dimensional structure 105 is formed of woven and/or non-woven fibers arranged in a network, layers, lattice, and/or scaffolding. In such cases, the void volume includes the open space between the fibers forming the overall three dimensional structure 105. For example, the first expandable cell culture substrate 100 can be macroscopic, where the three-dimensional structure 105 includes an x-dimension, a y-dimension, and a z-dimension, with each dimension independently between about 2.5 mm and about 10 mm, but the void volume can constitute microscopic pores or microscopic open spaces within the material forming the general three-dimensional structure 105. The void volume may filled with a liquid, making the overall appearance of the first expandable cell culture substrate 100 seem to be solid.

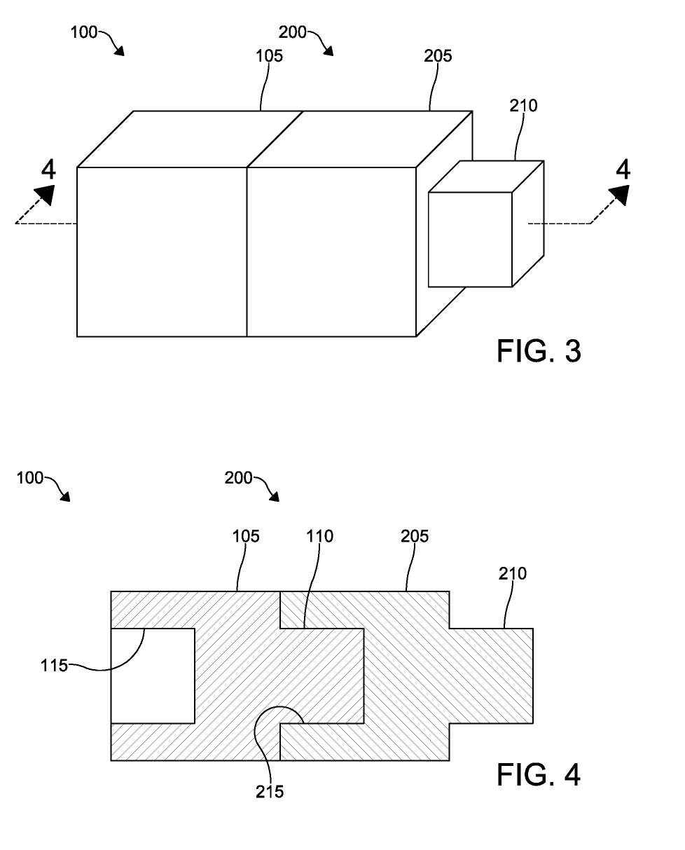

Continuing to FIGS. 3 and 4, the first expandable cell culture substrate 100 is shown coupled to a second expandable cell culture substrate 200, where the second expandable cell culture substrate 200 includes a three-dimensional structure 205 having a male coupling point 210 and a female coupling point 215. As shown, the first expandable cell culture substrate 100 and the second expandable cell culture substrate 200 are substantially identical. However, these substrates 100, 200 could be different in various ways, including the use of different materials or properties of the respective three-dimensional structures 105, 205.

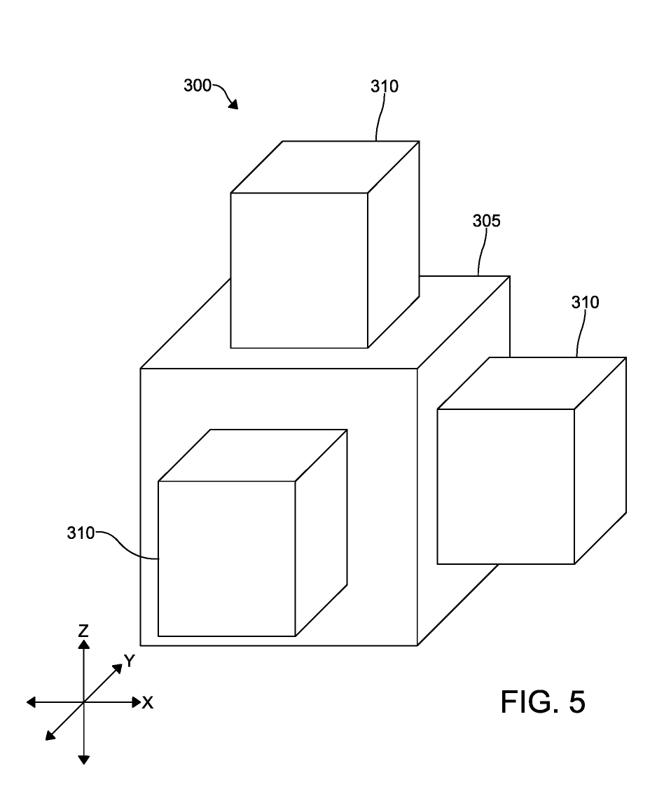

A third expandable cell culture substrate 300 is shown in FIG. 5. The three-dimensional structure 305 is generally cuboid and includes three male coupling points 310. Although not shown, there can be one or more female coupling points on the remaining faces of the cuboid three-dimensional structure 305. The three male coupling points 310 radiate from the three-dimensional structure 305 in three dimensions, allowing the third expandable cell culture substrate 300 to be coupled to another expandable cell culture substrate in one or more of the depicted x-, y-, and z-axes. It is also possible to expand the third expandable cell culture substrate 300 by coupling with the first expandable cell culture substrate 100 shown in FIGS. 1 and 2, and/or expand the third expandable cell culture substrate 300 by coupling with pre-coupled first and second expandable cell culture substrates 100, 200 shown in FIGS. 3 and 4.

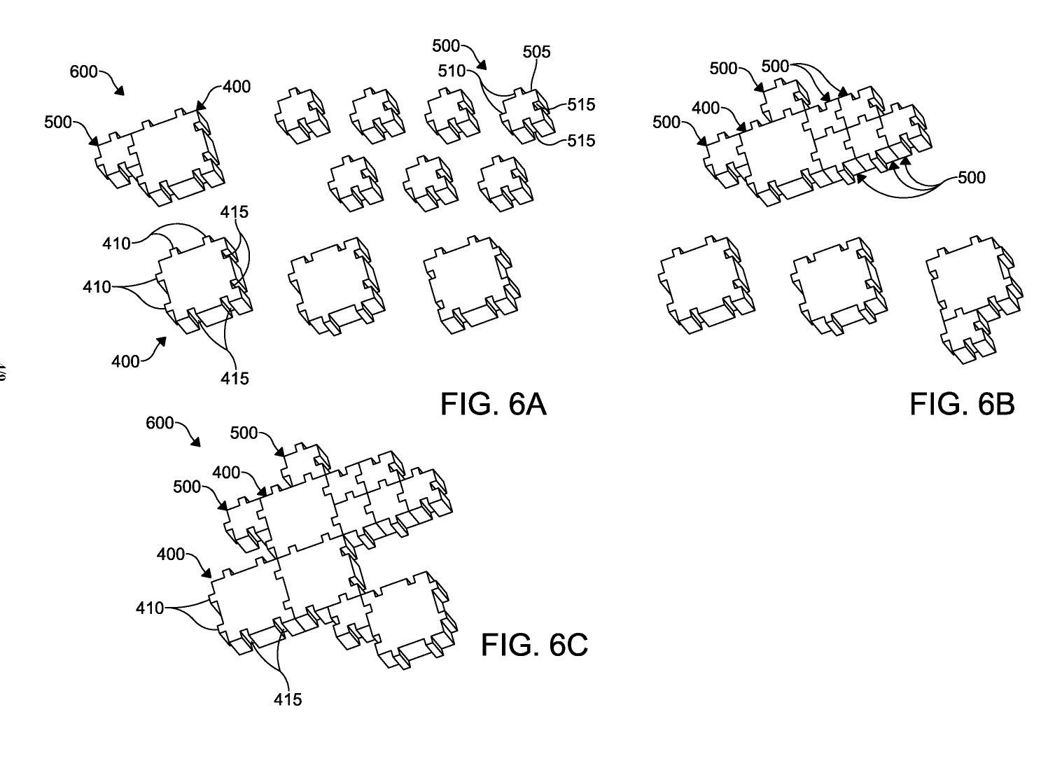

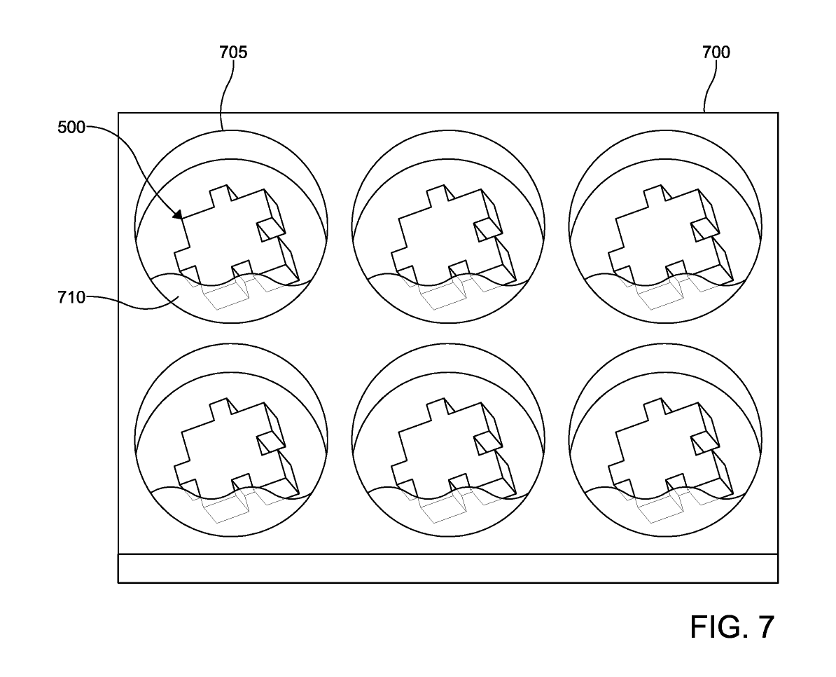

Assembly of various expandable cell culture substrates is shown in FIG. 6. Panel A shows several separate embodiments of a fourth expandable cell culture substrate 400 including a three-dimensional structure 405 having a plurality of coupling points, where the plurality of coupling points includes four male coupling points 410 and four female coupling points 415. The three-dimensional structure 405 of each fourth expandable cell culture substrate 400 has a void volume configured to accommodate a cultured cell. The male and female coupling points 410, 415 are complementary where respective pairs of the male and female coupling points 410, 415 permit coupling of the three-dimensional structures 405, 505 with each other. Also shown in panel A are several separate embodiments of a fifth expandable cell culture substrate 500, including a three-dimensional structure 505 having a plurality of coupling points, where the plurality of coupling points includes two male coupling points 510 and two female coupling points 515. The three-dimensional structure 505 of each fifth expandable cell culture substrate 500 has a void volume configured to accommodate a cultured cell. The male and female coupling points 510, 515 are complementary and permit coupling of the three-dimensional structures 505 with each other. The fifth expandable cell culture substrate 500 is also depicted as about one-quarter the size of the fourth expandable cell culture substrate 500 Also shown at 600 in panel A is a fourth expandable cell culture substrate 400 coupled to a fifth expandable cell culture substrate 500. The fourth and fifth expandable cell culture substrates 400, 500 at 600 can be provided as pre-coupled three-dimensional structures 405, 505, which can later be separated or further coupled with separate or pre-coupled fourth and fifth expandable cell culture substrates 400, 500. Turning to panel B, multi-dimensional coupling between the fourth expandable cell culture substrate 400 and several fifth expandable cell culture substrates 500 is shown. Turning to panel C, multi-dimensional coupling between several fourth expandable cell culture substrates 400 is shown, where further multi-dimensional coupling with several fifth expandable cell culture substrates 500 is shown. Panel C provides an example of how the fourth and fifth expandable cell culture substrates 400, 500 can be configured as interlocking blocks to form a tessellation. The different sizes of the fourth and fifth expandable cell culture substrates 400, 500 can be used to tailor cell culture conditions based on propagation and/or location of cells. The different sizes also allow control with respect to shaping a coupled structure of multiple substrates 400, 500, including the separation of substrates 400, 500 to dispensing into other cell culture vessels or bioreactors. Although not shown, the fourth and fifth expandable cell culture substrates 400, 500 could include coupling points in a third dimension, allowing expansion along the z-axis in a fashion similar to that depicted for the third expandable cell culture substrate 300 shown in FIG. 5.

With reference now to FIG. 7, a six-well cell culture plate 700 is shown with each well 705 including a fifth expandable cell culture substrate 500 (as per FIG. 6) along with growth media 710. The fifth expandable cell culture substrate 500 can be submerged in the growth media 710, where the growth media 710 can be changed as necessary or desired. Alternatively, the fifth expandable cell culture substrate 500 can be moved to a new cell culture vessel or bioreactor including fresh cell culture media or a different cell culture media. Each well 705 of the six-well cell culture plate 700 can also include different growth media 710 or additives to the growth media 710, allowing for selection or screening of cells growing on each fifth expandable cell culture substrate 500.

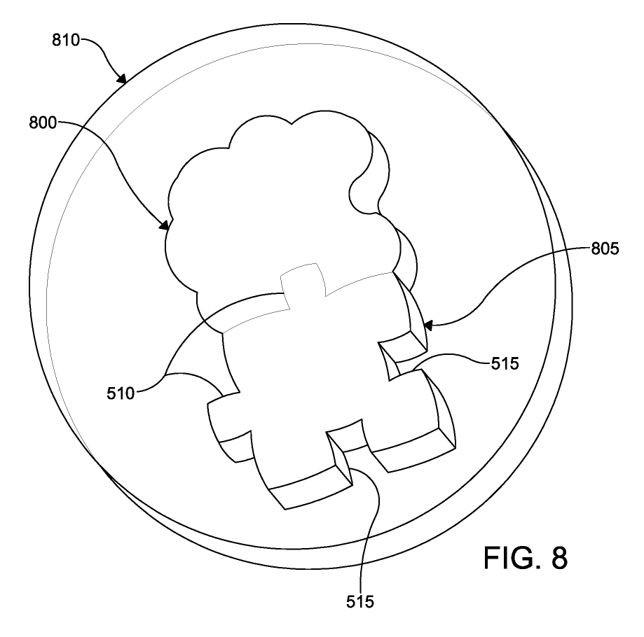

A representation of an original expandable cell culture substrate 800 with a later coupled expandable cell culture substrate 805 in a cell culture plate 810 is shown in FIG. 8. Each of the original and later coupled expandable cell culture substrates 800, 805 are substantially identical to the fifth expandable cell culture substrate 500 shown in FIG. 6. Here, the original and later coupled expandable cell culture substrates 800, 805 include three-dimensional structures formed of biodegradable material. The original expandable cell culture substrates 800 is at or near saturation with cells, where the cells are proliferating into and/or migrating into the later coupled expandable cell culture substrate 805. The overall shape of the original expandable cell culture substrate 800 is depicted as less distinct, as degradation of the three-dimensional structure thereof over time can result in a more amorphous character. The later coupled expandable cell culture substrate 805 is shown generally retaining its three-dimensional structure form, where the male and female coupling points 510, 515 remain substantially defined and distinct as cells proliferate and expand into the substrate 805.

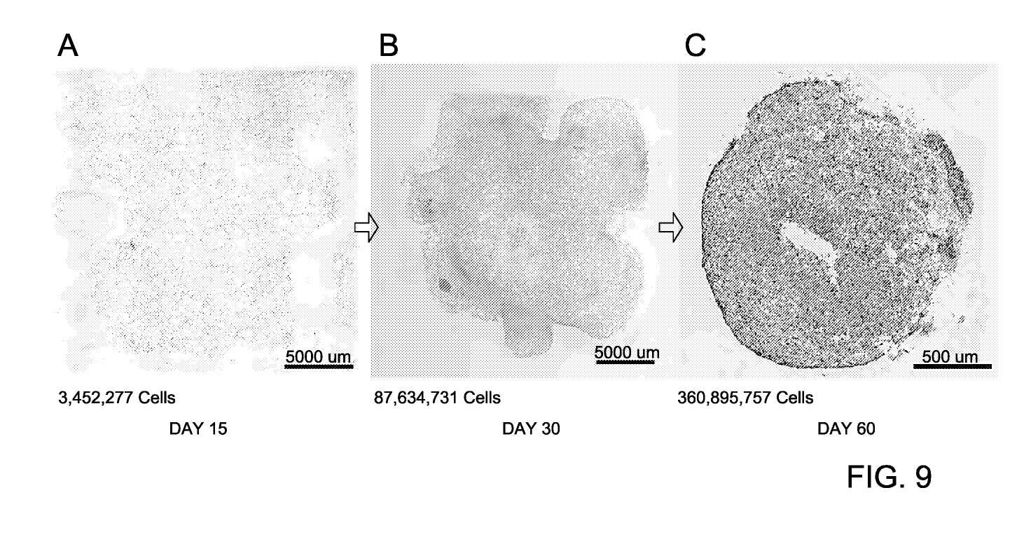

FIG. 9 provides a series of photomicrographs of an expandable cell culture substrate having cells proliferating in the void volume thereof. Panel A shows about 3.5 million cells at day 15, panel B shows about 88 million cells at day 30, and panel C shows about 361 million cells at day 60. It should be noted that the cells are proliferating in three-dimensions throughout the expandable cell culture substrate. In this way, another expandable cell culture substrate can be coupled thereto in any of the x-, y-, and z-axes to provide additional surface area for cells. Panels A and B include a 5,000 micrometer reference bar, whereas panel C is at greater magnification and includes a 500 micrometer reference bar.

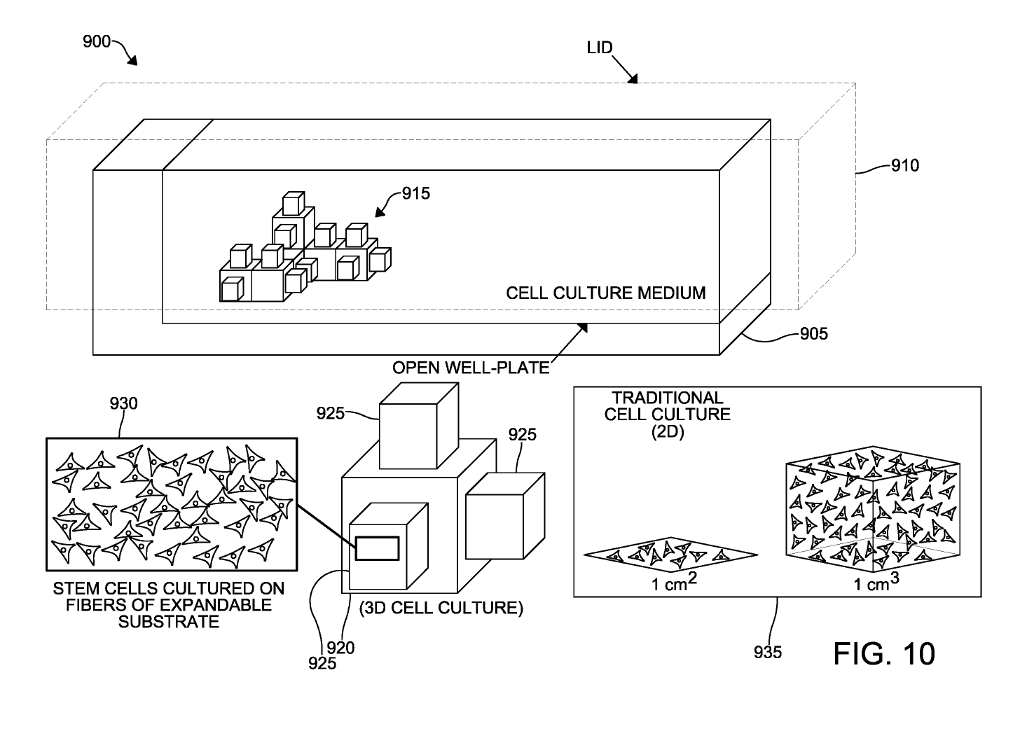

FIG. 10 is a schematic of an elongated cuboid cell culture apparatus 900 having an open well plate 905 and a lid 910. A group of coupled expandable cell culture substrates is shown at 915, where the expandable cell culture substrates are coupled in three-dimensions, expanding the available growth area for cells in the x-, y-, and z-axes. A close-up view of a single expandable cell culture substrate is shown at 920, which can include male coupling points 925, as shown, as well as complementary female coupling points on the other three faces (not shown) of the substantially cuboid three-dimensional structure. An inset 930 of the expandable cell culture substrate 915 depicts stem cells proliferating within the substrate 915, where the stem cells can adhere to fibers of the three-dimensional structure. The cell culture apparatus 900 can be fluidly coupled to a circulation system (not shown) allowing perfusion of cell culture media and movement of cell culture media through the group 915 of coupled expandable cell culture substrates. As can be seen, there is space within cell culture apparatus 900 to further couple additional expandable cell culture substrates depending on cell propagation rates and densities throughout various portions of the group 915 of coupled expandable cell culture substrates. Inset at 935 contrasts the difference in available surface area and cell density for stem cells grown on traditional two-dimensional culture plates versus expandable cell culture substrates according to the present technology.

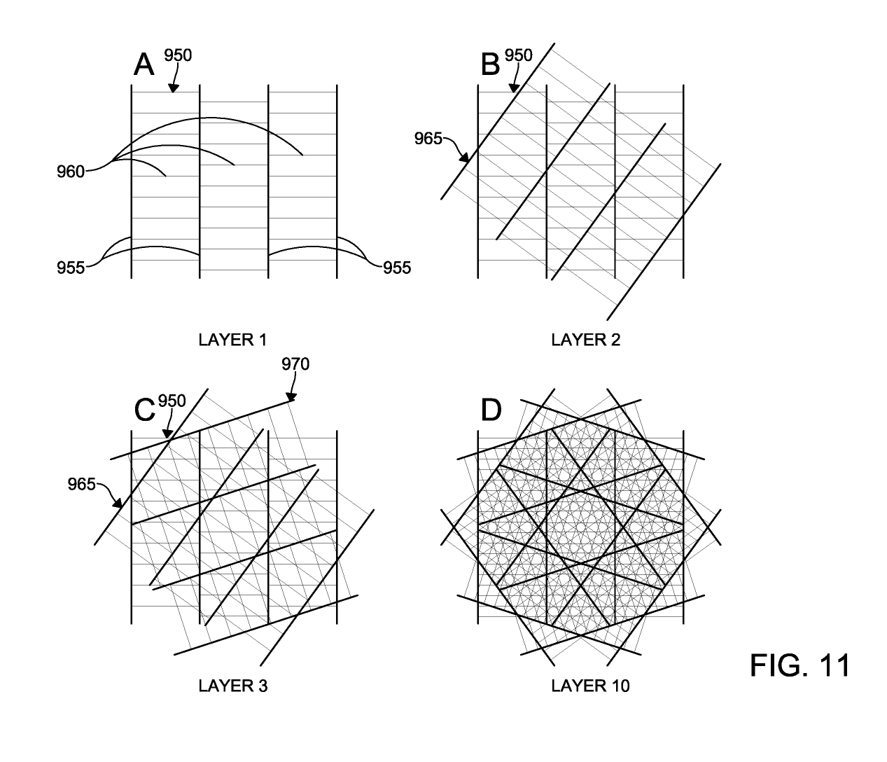

One method of making an expandable cell culture substrate using multiple layers of material is shown in FIG. 11. Panel A shows a first layer 950 including first fibers 955 that are coupled together by second fibers 960. As shown, multiple second fibers 960 can couple the first fibers 955 together. The first fibers 955 and the second fibers 960 can be different sizes and/or thicknesses and can be made of different materials. Panel B shows a second layer 965 placed over the first layer 950. As shown, the second layer 965 can be formed in the same way as the first layer 950; however, it should be appreciated that an entirely different type of layer can be used, including different sizes, thickness, and types of materials. The second layer 965 is also shown placed over the first layer 950 at a different orientation from the first layer 950, where the second layer 965 is rotated clockwise relative to the first layer 950. Panel C shows a third layer 970 placed over the second layer 965 and further rotated clockwise. The addition of layers can continue with additional clockwise rotations, where panel D shows a total of ten layers. As can be seen, the network of first fibers 955 and second fibers 960 provides a tortuous path through the layers, where the layers provide a dense lattice work of fibers with space therebetween providing a void volume for cells. Additional layers beyond the ten shown in panel D can be provided, or the multi-layer structure in panel D can be shaped to fashion a three-dimensional structure with one or more coupling points, such as by die-cutting the multi-layer structure.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

1. An expandable cell culture substrate comprising:
a three-dimensional structure including a coupling point, the three-dimensional structure including a void volume configured to accommodate a cultured cell, and the coupling point configured for reversible coupling to another three-dimensional structure.

2. The expandable cell culture substrate of claim 1, wherein the three-dimensional structure and the another three-dimensional structure are the same.

3. The expandable cell culture substrate of claim 1, wherein the three-dimensional structure and the another three-dimensional structure are different.

4. The expandable cell culture substrate of claim 1, wherein the three-dimensional structure includes a plurality of coupling points.

5. The expandable cell culture substrate of claim 4, wherein the plurality of coupling points includes a first coupling point and a second coupling point.

6. The expandable cell culture substrate of claim 5, wherein the first coupling point and the second coupling point are the same.

7. The expandable cell culture substrate of claim 5, wherein the first coupling point and the second coupling point are different.

8. The expandable cell culture substrate of claim 5, wherein the first coupling point is a male coupling point and the second coupling point is a female coupling point.

9. The expandable cell culture substrate of claim 8, wherein the male coupling point and the female coupling point are complementary.

10. The expandable cell culture substrate of claim 1, further comprising a plurality of three-dimensional structures, each of the three-dimensional structures including a coupling point, each of the three-dimensional structures including a void volume configured to accommodate a cultured cell, and the coupling point of each three-dimensional structure configured for coupling to another three-dimensional structure.

11. The expandable cell culture substrate of claim 10, wherein the plurality of three-dimensional structures includes one three-dimensional structure coupled to another three-dimensional structure.

12. The expandable cell culture substrate of claim 1, wherein the three-dimensional structure includes a porous material having open cavities.

13. The expandable cell culture substrate of claim 1, wherein the three-dimensional structure includes a reticulated foam.

14. The expandable cell culture substrate of claim 1, wherein the three-dimensional structure includes a mesh-like material.

15. The expandable cell culture substrate of claim 1, wherein the three-dimensional structure comprises a member selected from the group consisting of: poly(lactic-co-glycolic acid); polylactic acid; polycaprolactone; hyaluronic acid; chitosan; alginate; acellular matrix; polyurethane; polyurethane-polyether; polycarbonate; and combinations thereof.

16. The expandable cell culture substrate of claim 1, wherein the three-dimensional structure includes an additive selected from the group consisting of: silver; gold; fibronectin; chitosan; poly-L-lysine; hyaluronic acid; a growth factor; laminin; fibrin; and combinations thereof.

17. The expandable cell culture substrate of claim 1, wherein the three-dimensional structure includes an x-dimension, a y-dimension, and a z-dimension, each dimension independently between about 2.5 mm and about 10 mm.

18. The expandable cell culture substrate of claim 1, wherein the three-dimensional structure includes a plurality of coupling points, the plurality of coupling points includes a first coupling point and a second coupling point, the first coupling point and the second coupling point are different, the three-dimensional structure includes an x-dimension, a y-dimension, and a z-dimension, each dimension independently between about 0.25 cm and about 1 cm, and the three-dimensional structure comprises a member selected from the group consisting of: poly(lactic-co-glycolic acid); polylactic acid; polycaprolactone; hyaluronic acid; chitosan; alginate; acellular matrix; polyurethane; polyurethane-polyether; polycarbonate; and combinations thereof.

19. The expandable cell culture substrate of claim 1, further comprising a cell within the void volume.

20. The expandable cell culture substrate of claim 19, wherein the cell is a stem cell.

21. A method of culturing a cell comprising:
providing the cell with an expandable cell culture substrate according to claim 1.

22. The method of claim 21, further comprising coupling another expandable cell culture substrate to the expandable cell culture substrate.

23. The method of claim 21, further comprising perfusing the expandable cell culture substrate with cell culture media.

24. The method of claim 21, wherein the cell is a stem cell.

25. A method of making an expandable cell culture substrate comprising:
providing a layer of material;
adding at least one additional layer of material to the provided layer of material, thereby forming a plurality of layers of material, the plurality of layers of material providing a three-dimensional structure including a void volume configured to accommodate a cultured cell; and
shaping the three-dimensional structure to form a coupling point configured for reversible coupling to another three-dimensional structure.

26. The method of claim 25, wherein the layer and the additional layer are the same.

27. The method of claim 25, wherein the layer and the additional layer are different.

28. The method of claim 25, wherein the at least one additional layer is added at an orientation that is different from the provided layer.

29. The method of claim 25, wherein the provided layer, the at least one additional layer, or both the provided layer and the at least one additional layer includes fibers.

30. The method of claim 29, wherein the fibers including a plurality of first fibers and a plurality of second fibers, the first fibers being thinner than the second fibers.

31. The method of claim 30, wherein the first fibers are coupled to the second fibers.

32. The method of claim 25, wherein shaping the three-dimensional structure includes die-cutting the three-dimensional structure.

33. The expandable cell culture substrate of claim 8, wherein the male coupling point is a protrusion and the female coupling point is a recess, the protrusion configured to substantially fill the recess when the three-dimensional structure is coupled to the another three-dimensional structure.

34. The expandable cell culture substrate of claim 1, wherein the male coupling point is a cuboid protrusion and the female coupling point is a cuboid recess.

35. The expandable cell culture substrate of claim 1, wherein the three-dimensional structure is comprised by a plurality of three-dimensional structures, the plurality of three-dimensional structures configured to form a tessellation in at least two dimensions when coupled together.

36. The expandable cell culture substrate of claim 1, wherein the three-dimensional material includes acellular matrix.
